(19) United States
(12) Patent Application Publication
(10) Pub. No.: US 2004/0225198 A1
(43) Pub. Date: Nov. 11, 2004

(54) Method for treatment of neurodegenerations

(76) Inventor: Natalia Veniaminovna Kazantseva, Moscow (RU)

Correspondence Address: Natalia Veniaminovna Kazantseva, University Pr 5 480 Moscow, Moscow, 117296, RU

(21) Appl. No.: 10/276,232

(22) PCT Filed: Mar. 4, 2002
(86) PCT No.: PCT/RU02/00070

(30) Foreign Application Priority Data
Mar. 5, 2001 (RU) 2001105995

(51) Int. Cl. A61B 5/00

(52) U.S. Cl. 600/300

(57) ABSTRACT

The present invention relates for use in medicine, more specifically neurology, to a method for treatment of neurode generation and autoimmune diseases. The innovation is intended to increase the efficacy of treatment of cerebral neural degenerative diseases and multiple sclerosis using minimized hyperbaric therapy combined with metabolic antioxidants (Pat. No. 2,140,247, Russia). To achieve this goal chamber sessions at pressure from 1.03 to 1.1 ATA against the background of antioxydants inducing mithochondrial oxidation were combined with Cyclosporine-A at a dose below therepeutical. Treatment efficacy was controlled using common methods and monitoring immune status dynamics, particularly immune status suppression following its activation, whereupon Cyclosporine-A dose was gradually reduced till complete withdrawal. A combination of minimized hyperbaric therapy and cyclosporine-A administration intensifies the treatment effect of each component. The previously used minimized hyperbaric method failed to totally or substantially restore the functions impaired by a secondary progressive MS or motor functions impaired by side amiotrophic selerosis. Timely application of the proposed method for treatment of neutral degeneration and MS would help preserve work ability and improve patient's quality of life.

The present invention relates for use in medicine, more specifically neurology, to a method for treatment of degenerative cerebral disorders and autoimmune diseases.

Known in the prior art is minimized hyperbaric treatment with pressure in chamber from 1.03 to 1.1 ATA combined with metabolic action antioxidants used for treatment of cerebral degenerative diseases and multiple sclerosis (MS) [Pat. No. 2 140 247, Russia].

Equally known in the prior art is using cyclosporine-A as immune depressant during treatment of autoimmune diseases and organ transplantation. However cyclosporie-A use for treatment of MS showed no higher effect compared to other cytostatic agents, e.g. Azatiprine [Godkin D., E. Neurology, 1991, 41:980-985.]. It was also associated with a serious risk of potential side effects, e.g. hepatorenal and peripheral blood modifications, and oncological diseases [Rudge P., Koester J. C., Mertin J. et al., J. Neurol. Neurosturg Psychiatry, 1989, 52:559-565].

The closest to the proposed technique is the “Method for treatment of multiple sclerosis and cerebral degenerative disorders” (3). It is intended to increase the efficacy of treatment of cerebral degenerative disorders and multiple sclerosis using minimized hyperbaric treatment combined with metabolic antioxidants and immune modulators. It is achieved by treatment sessions in a pressure chamber at excessive pressure from 1.03 to 1.1 ATA combined with cyclosporine-A dose below therapeutical. Treatment effectiveness is monitored using common methods and dynamics of immune status, particularly its depression followed by activation whereupon cyclosporine-A dose is gradually lowered till complete withdrawal.

The essence of the innovation is to provide a new quality of treatment by using a minimum excess pressure in a pressure chamber to restore microcirculation combined with cyclosporine-A effect suspending autoimmune inflammation and lowering neurocyte apoptosis.

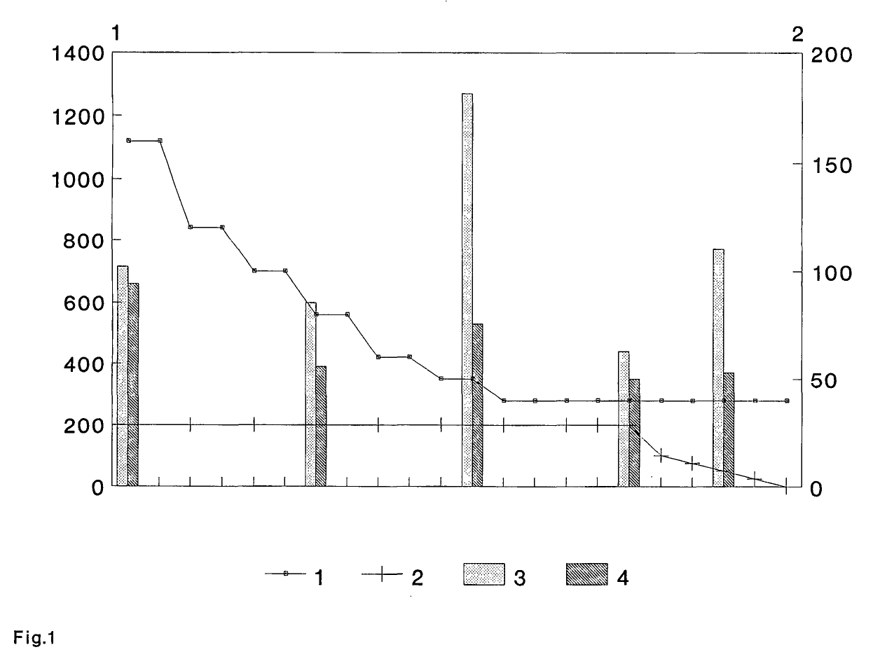

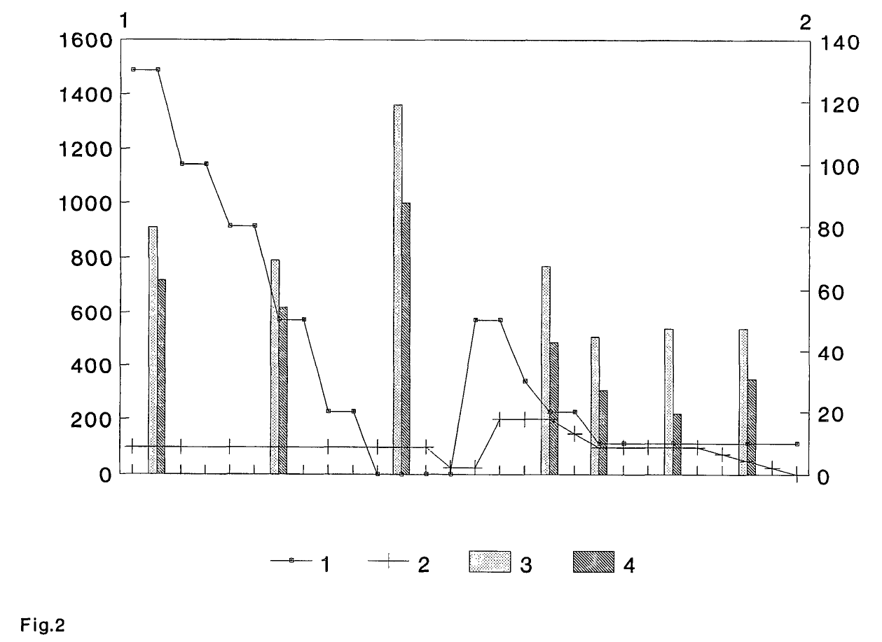

FIG. 1 shows the dynamics of clinical symptoms and immune parameters under minimized hyperbaric treatment combined with metabolic antioxidants and cyclosporine-A in patient J. (example 1). FIG. 2 shows the dynamics of clinical symptoms and immune status under minimized hyperbaric treatment combined with metabolic antioxidants and cyclosporine-A in patient M. (example 2).

The technique application is illustrated by the following examples.

### Example 1.

Patient J., male, 34 year old, with multiple sclerosis since he was 24 years old. Neurological symptoms were continuously aggravating within the last four years. Following Sinakten course in January 2000 the patient's condition continued to deteriorate. From January 2000 he received 25 mg of azatioprin four times a day, 2 mg of cyclodol three times a day, and 25 mg of amitriptyline two to three times a day.

We observed the patient since March 2000. The status examination showed a gross ataxia of cerebellum, wobbling in Romberg position with feet widely placed, tremor of protruded fingers, intentional tremor and missing during finger-nose and knee-heel tests. Gross oculomotor distortions, non-convergent, with spontaneous end-position nystagmus intensified during the eyeball side and front movements with a rotary component. Facial muscle dystonia and hyperkynetic twitching of extremities. The gait is ataxic with widely placed feet. Absent abdominals reflexes. Muscle strength is adequate. Foots clonus with inconstant double-sided Babinski signs. Imperative desire to urinate, sexual dysfunction. Impaired left eye vision. The patient is non-critical to his status, depressed, the intellectual status is lowered.

During the examination: Immune status: signs of activated autoimmune status are present. Blood test shows leukocytosis up to 13 thousand, ESR with a left-shifted formula. Urine test using Nechiporenko method showed leukocytosis up to 45 thousand per 1 mL.

EEG showed profound diffuse disturbances with focal epileptiform lesion in the right frontal temporal segments.

MRT showed focal demyelination in subcortex formations, cerebellum and brainstem and moderate cortical atrophy.

Eyeground: pale temporal segments of the optic disks with partial atrophy of the right optic disk.

Treatment: 100 mg Furagin three times a day during ten days and 100 mg Finlepsin twice a day. Following minimized hyperbaric treatment in a pressure chamber combined with 30 mg coenzyme Q10 and 50 mg pycnogenol and short course of dexarnethazone (0.2 mg/kg bodyweight daily preceding minimized hyperbaric session, gradually reducing the initial dose) the patient's status improved along with reduced ataxia, passive tremor and facial dystonia. Cyclodol and amitriptyline were canceled.

Notwithstanding azatioprin (cytostatic agent) the immune status retained the signs of active autoimmune inflammation against the background of some activation of cellular immune supression induced by minimized hyperbaric treatment.

Given the activity of autoimmune inflammation against the background of cytostatic administration, the patient was treated with cyclosporine-A at 2 mg/kg bodyweight with daily sessions of minimized hyperbaric treatment with pressure from 1.05 to 1.1 ATA combined with 30 mg Q₁₀ and 50 mg Picnogenol. Cyclosporine-A administration induced a noticeable and stable restoration of the lost functions. Neurological symptoms regress was observed during each barochamber session with the clinical effect growing from one session to another. Immediately upon cyclosporine-A administration the patient gradually (in 4 to 5 days) withdrew from azatioprin. The initial depression of the immune status (within two weeks) was followed by its activation, and upon 2 months of cyclosporine-A administration the repeated immune depression was reported that served a criterion for a gradual withdrawal of the medication. The patient remained stable during 6 months following the withdrawal of cyclosporine-A. The patient is stable in Romberg position, position tremor is absent, finger-nose test is performed with the expressed right-side intention in the vertical position, and left-side in the vertical position. The eye motion distortions have considerably diminished. Nystagmoid remains unstable at extreme position of eyeballs. Abdominal and plantar reflexes are induced, feet clonus is absent. Urination has stabilized. FIG. 1 shows the dynamics of neurological symptoms expressed in points (1), cyclosporine-A dose (2), T-lymphocyte content in blood (3) and first type T-helpers (4).

The patient with a steady progressing secondary postgradient multiple sclerosis who previously received hormone and cytostatic therapy combined with minimized hyperbaric treatment without a substantial improvement, evidenced an unexpected clinical effect of this method combined with cyclosporine-A.

### Example 2.

Patient M., male, 22 years old, has suffered from multiple sclerosis since he was 16 years old, had secondary progressive disease with exacerbation twice a year. We observed this patient since September 1998 and he was treated with minimized hyperbaric treatment combined with antioxdants. However this method failed to improve the status during exacerbation in August 1999. Despite the signs of autoimmune activation, the patient refused from a hormone therapy course.

Status: Wobbling in Romberg position, expressed ataxia, tremor of protruded fingers, intentional tremor and missing predominantly to the left side. Gross intention during a feet-knee test with worse results on the right side. Expressed oculomotor symptoms, distorted convergence, incomplete internal drive of the eyeball, limited eyeball motion in the upward direction. Horizontal nystagmus with a rotary component during the upward look. Upper left eyelid ptosis, myosis and enophtalm. Anisoreflexia more expressed on the right side. Paresis of the lower right extremity up to 3.5 points. Babinski signs on both sides, feet clonus more profound on the right side. The patient denies pelvis problems, and is slightly euphoric.

Eyeground: Pale temporal segments of the optic disks with partial atrophy of the right optic disk; vision normal.

MRT: Multifocal cerebral lesion — preventricular in the brainstem; focal demyelination in the cerebellum and oblongata; negative dynamics compared to 1997.

In November 1999 the patient took a course of cyclosporine-A (2 mg/kg) with daily barochamber sessions under pressure from 1.06 to 1.08 ATA combined with 30 mg Q₁₀ and 50 mg picnogenol. Cyclosporine-A administration restored the lost functions during each barochamber session with practically total regression of neurological symptoms upon two weeks treatment. Against the background of expressed activation of the immune status, the patient stopped taking cyclosporin-A (with no barotherapy sessions on week ends). It aggravated oculomotor distortions and resulted in complete left-side ophtalmoplegia.

The patient resumed taking cyclosporine-A at 2 mg/kg dosage subsequently growing up to 4 mg/kg bodyweight, where upon a repeated symptoms regress was reported during barochamber sessions. The patient restored the lost body functions, and one month later cyclosporine-A was gradually withdrawn. FIG. 2 shows the dynamics of neurological symptoms (1), cyclosporine-A dose (2), T-lymphocyte content in blood (3) and T-helpers (4).

Observation over the patient within twelve months following a complete withdrawal of cyclosporine-A evidenced a stable and long-tern treatment effect.

### Example 3.

Patient A., female, 58 years old, with a two-year history of side amiotrophic lateral sclerosis with a gradually increasing impaired motor function along with bulbary distortions, with fasciculations, brainstem dysartria, impaired swallowing and expressed salivation. Minimized hyperbaric treatment combined with metabolic antioxidants (Q₁₀ and Picnogenol) resulted in less expressed bulbary distortions without a noticeable improvement of motor functions. The patient had psoriasis with pronounced eruption (for treatment of which cyclosporine-A is also used) that inclined us to use low dosage of cyclosporine-A combined with barochamber sessions. The first barochamber sessions against the background of cyclosporine-A resulted in a significant growth of force in the patient's extremities, particularly atrophic hand muscles thenar and hypothenar (scored in points) from point 1 when the patient failed to perform the thumb contraposition to 1-5 points with a noticeable increase of the muscle mass. The patient was satisfied with the treatment results and stopped visiting the clinic making phone calls only.

No similar effects have been reported in literature, and their existence could hardly be expected.

The proposed method for treatment of degenerative cerebral diseases hardly seems evident to neurologists experienced in the field. In particular, under the well-known method for cyclosporine-A use to slow down progressive multiple sclerosis, immune suppression is achieved following a long-term administration and is accompanied by a withdrawal phenomenon. It is reported in literature that administration of cyclosporine-A dose of 5 mg/kg bodyweight on a daily basis during two years failed to show a reliable effect for multiple sclerosis treatment, while a dose of 8 mg/kg during two years induced expressed complications associated with side effects [Rudge P., Koester J. C., Mertin J., et al. J. Neurol. Neurosurg Psychiatry, 1989, 52:559-565.].

We propose administration of cyclosporine-A at a dose from 1 to 4 mg/kg bodyweight during 1 to 3 months that shall be safe for a patient. Moreover, isolated use of cyclosporine-A is not accompanied by regressing neurological symptoms in patients with secondary progressing MS. We have for the first time evidenced a restoration of lost functions during secondary progressing MS; no similar facts have been reported in literature. There are no reports in literature of restored motive functions and increased muscle mass achieved during treatment of side amiotrophic lateral sclerosis.

Neurologists specializing in treatment of neural degeneration seem to be absolutely unaware of potential use of low-dosage cyclosporine-A combined with minimized hyperbaric treatment for a long-term restoration of immune status. No reports in literature are devoted to a possible restoration of immune regulation by using low-dosage of cyclosporine-A.

A combination of minimized hyperbaric treatment and cyclosporine-A administration intensifies the treatment effect of each component due to specific properties of cyclosporine-A. The latter prevents neurocyte apoptosis as a result of inhibited opening of non-specific pore in mitochondria and stabilizing cell membrane status [Nicolli A., et al., J. Biol. Chem. 1996.v.271, No. 4, p.2185-2192; Gogvadze V., et al. FEBS, 1993, 333(3):334-338.]

The proposed treatment method is characterized by a highly innovative nature and may have far-reaching medical, social and economic implications. The previously used minimized hyperbaric therapy method failed to totally or substantially restore the functions impaired by a secondary progressive multiple sclerosis or motor functions impaired by side amiotrophic lateral sclerosis. Timely application of the proposed method for treatment of neural degeneration and multiple sclerosis would help preserve work ability and improve the patient's quality of life. The proposed method could be broadly used in neurological and therapeutical wards of hospitals, outpatient clinics and rehabilitation centers.

1. A method for treatment of neurodegenerations is a combination of general impacts by excessive pressure from 1.03 to 1.1 ATA in a pressure chamber during 20 minutes daily sessions and administration of medications inducing mitochondrial oxidation. Pressure chamber sessions are combined with administration of cyclosporine-A. As a result the immune status is suppressed following its activation, whereupon cyclosporine-A dose is gradually reduced till complete withdrawal.
